(19) United States
(12) Patent Application Publication
(10) Pub. No.: US 2020/0016107 A1
(43) Pub. Date: Jan. 16, 2020

(54) COMPOSITION AND METHOD FOR THE TOPICAL TREATMENT OF SEVERE ACNE

(71) Applicant: CENTRO INTTERNACIONAL DE COSMIATRÍA, S.A.P.I. DE C.V., Ciudad De México (MX)

(72) Inventor: Sergio NACHT, Las Vegas, NV (US)

(73) Assignee: CENTRO INTERNACIONAL DE COSMIATRÍA, S.A.P.I. DE C.V., Ciudad De México (MX)

(21) Appl. No.: 16/491,338

(22) PCT Filed: Mar. 7, 2018
(86) PCT No.: PCT/MX2018/000017

§ 371 Date: Sep. 5, 2019

(30) Foreign Application Priority Data
Mar. 8, 2017 (MX) MX/A/2017/003003

(51) Int. Cl. A61K 31/203; A61K 31/60; A61K 31/65; A61K 31/327; A61K 31/78; A61P 17/10; A61K 9/14; A61K 9/00

(52) U.S. Cl. CPC A61K 31/203; A61K 31/60; A61K 31/65; A61K 9/0014; A61K 31/78; A61P 17/10; A61K 9/146; A61K 31/327

(57) ABSTRACT

The invention consists of the simultaneous use of the two stereoisomeric forms of retinoic acid with or without the addition of an antibacterial agent effective against Propionibacterium acnes in a cosmetically suitable topical formulation. These formulations may also contain a polymeric system that provides a gradual release of one or more of the active ingredients to obtain extended therapy with less skin irritation.

### BACKGROUND OF THE INVENTION

### Acne Vulgaris Pathogenic Factors

As known, for acne to occur there must be three abnormal factors simultaneously:

A high secretion of the sebaceous gland, an abnormal keratinization of the walls of the pilosebaceous follicle and the presence of a facultative anaerobic bacterium, Propionibacterium acnes (P. acnes), that multiplies in the sebaceous gland feeding on triglycerides and releasing fatty acids that generate an inflammatory process.

### Therapeutic Strategies

All effective topical therapies are aimed at controlling one or more of these factors, as this results in an improvement of the acne condition.

Reduction in sebaceous secretion: The sebaceous gland is under hormonal control, particularly androgens and especially testosterone. Therefore, a reduction in androgen secretion reduces the activity of the sebaceous gland. Systemic estrogen administration has this effect, but, for obvious reasons, this type of medication is only acceptable for treating women since in men it has unacceptable feminizing side effects.

Oral administration of 13-cis-retinoic acid (Accutane in the US) has a profound effect on the sebaceous gland since it almost completely suppresses this secretion and thus causes a marked improvement in almost 100% of patients.

However, the side effects are severe and range from severe dryness of the skin, lips and even eyes to acute teratogenic effects in pregnant women. This makes this therapy reserved for very severe acne (conglobata) and in cases where other therapeutic forms have not worked. In addition, this therapy requires the simultaneous use of two forms of contraceptives to prevent pregnancy, which is not acceptable for many women for religious or psychological reasons.

Normalization of keratinization of the follicular wall: There are therapeutic agents that lead to this result, from Salicylic Acid, Sulfur (with or without Resorcinol)and retinoids such as all-trans-retinoic Acid, Adapalene, Tazarotene or Retinol.

Benzoyl Peroxide also has a minor keratolytic action.

Reduction of bacterial flora: This can be obtained either by oral or topical administration of antibiotics or by topical administration of some bactericidal compounds. However, it is important that the active compounds have a significant solubility (or partition coefficient) in the sebaceous secretion since that is where P. acnes dwells and multiplies. That is why tetracyclines are the antibiotics of choice (oral or topical) and Benzoyl Peroxide is the most effective topical bactericide.

In the World Dermatological Market there is a large number of topical products for the treatment of acne that act on one or two of the pathogenic factors of this condition. That is, they treat either the abnormal keratinization of the pilosebaceous follicle, the bacterial proliferation or both, but they have no effect on sebaceous secretion which is the initial and most important factor in this process since this secretion is what results in the adhesion of the Pilosebaceous duct wall cells preventing their normal desquamation. This results in the “plugging” of the duct generating comedones (open or closed) that are the initial acne lesion. These comedones provide a favorable field for the development of P. acnes, since this is a facultatively anaerobic bacterium, which proliferates in the closed follicle and initiates the inflammatory process.

### DETAILED DESCRIPTION OF THE INVENTION

The invention described herein can provide for the first time an effective and simultaneous topical treatment against all THREE PATHOGENIC FACTORS of acne.

These compositions and method of treatment result in high effectiveness with virtually no side effects and, since the application of 13-cis-retinoic acid (iso-tretinoin) is only topical and not systemic, this avoids possible teratogenic effects and significantly reduces the excessive dryness of mucous membranes and skin tissues.

### Description of the Invention

The invention consists of a topical formulation for the treatment of acne, in particular the most severe type of inflammatory acne.

The formulation contains 13-cis-retinoic acid in concentrations from 0.001% to 0.25% (preferably between 0.005% to 0.15%) and another retinoid with keratolytic therapeutic action in acne, especially but not restricted to all-trans retinoic acid in concentrations from 0.001% to 0.25% (preferably between 0.025% to 0.10%) in a cosmetically acceptable aqueous vehicle, containing suitable antioxidants and preservatives to protect retinoids against oxidation. This second retinoid can also be substituted for Salicylic Acid in concentrations between 0.5 and 5%, preferably between 1 and 2%.

The formulations described in the previous paragraph may also contain an antibiotic capable of controlling P. acnes, like clindamycin in concentrations of 0.5% to 3%, preferably between 1% and 2%.

The formulations described in the previous paragraph in which the antibiotic used is erythromycin or some other tetracycline in concentrations between 1% and 5%.

The formulations in the previous paragraph in which the antibiotic is replaced by Benzoyl Peroxide in concentrations between 2.5% and 10%.

The formulations of the preceding paragraphs in which all-trans-retinoic acid is substituted by Adapalene or Tazarotene in concentrations between 0.001% and 0.5%, preferably between 0.025% and 0.3%.

The formulations of the preceding paragraphs in which one or more of the active ingredients are included in polymeric particles such as those described in U.S. Pat. No. 5,145,675 and 9,149,490 to obtain a gradual and extended release of the active principle (s) included in the polymer system, said polymer particles are mainly composed of crosslinked polystyrene or polymethacrylates, more commonly divinylbenzene for the polystyrene polymer and ethylene glycol dimethacrylate for polymethacrylates. The polymeric particles have a particle size in the range of 5 to 300 μm, more commonly in the range of 10 to 25 μm; and a pore diameter and volume between 0.3 to 4 cm³/g, more commonly 2.0 cm³/g. This results in superior therapeutic effects with a total reduction in many cases in the side effects, especially topical irritation. This also allows reducing the dosage since in many cases a daily application is sufficient.

Method for the topical treatment of acne, especially severe inflammatory acne with topical formulations containing 13-cis-retinoic acid, and another retinoid with keratolytic action that can be all-trans-retinoic acid, Adapalene, Tazarotene or other or Salicylic Acid. The formulation may also contain an antibiotic such as Clindamycin, Erythromycin, Tetracycline, etc. or a bactericidal agent such as Benzoyl Peroxide.

One or more of the active ingredients may be included in a polymeric system suitable for obtaining a slow and extended release resulting in high efficacy with less skin irritation.

### Formulations

Formula 1

| | |
|---|---|
| Tretinoin USP^{1,2} | 0.12% |
| Isotretinoin¹ | 0.12% |
| Clindamycin³ | 1.20% |
| Carbomer 934P, NF | 0.80% |
| Glycerin USP | 12.50% |
| Propylene glycol | 4.60% |
| PPG-20 Methylglucose ether distearate | 4.75% |
| Cyclomethicone and Dimethicone Copoliol | 2.30% |
| Crosslinked Acrylate Polymer (Microsponge) | 8.85% |
| Trolamine NF | 0.40% |
| BHT | 0.02% |
| Disodium edetate | 0.01% |
| Benzyl alcohol | 0.90% |
| Deionized water | 63.48% |

Notes:
¹Tretinoin and isotretinoin can be dispersed in the vehicle or pre-incorporated into the acrylate polymer
²Tretinoin can be substituted for adapalene or tazarotene, or salicylic acid
³Clindamycin may be substituted for erythromycin or tetracycline

### Control Formulas

Formula 2: Identical to Formula 1 but without isotretinoin
Formula 3: Identical to Formula 1 but without tretinoin
Formula 4: Identical to Formula 1 but without the antibiotic

### Alternative Formulas in Double Chamber Containers

All Retinoids are unstable in the presence of oxidizing agents such as Benzoyl Peroxide or acids such as Glycolic Acid or Salicylic Acid.

However, these compounds can be administered together with retinoids as long as they are kept separate until they are dispensed on the skin of the acne affected area.

There are new double packages (double chamber) in which the formulation is packed containing the retinoids in one chamber and the other containing the oxidizing agent or acid in the other. Each of these chambers is provided with a pump that can be operated independently or simultaneously to dispense the formulas.

The mechanical design of these containers requires that for their optimal functioning the active compounds must be formulated in vehicles of the same or very similar composition so that the formulas in each chamber have the very close to the same specific viscosity or density.

### Examples of Realization

The following formulas are examples of this type of formulations that can be used in double containers.

Formula 5-A
Active principles:

| | |
|---|---|
| Tretinoin USP | 0.12% |
| Isotretinoin | 0.12% |
| Vehicle | |
| Starch Phosphate | 14.40% |
| Bentonite | 7.75% |
| Zinc Oxide | 4.25% |
| Polyethylene glycol 400 | 5.00% |
| Oleth-10 | 2.20% |
| Menthol | 0.15% |
| Sodium Hexametaphosphate | 0.03% |
| Methylparaben | 0.18% |
| Propylparaben | 0.02% |
| Deionized water q.s. | 100.00% |

Formula 5-B
Active principles

| | |
|---|---|
| Benzoyl Peroxide Hydrate (70%) | 8.57% |
| | (instead of the |
| | antibiotic) |

Note:
This results in a final BPO concentration of 6.00% (nominal 5% plus 20% overload). Likewise, 4.2% BPO can be used to obtain a nominal final concentration of 2.5% or 15.5% to obtain a nominal final concentration of 10%)
Vehicle: As in Formula 5-A.

### Clinical Study

The efficacy of these formulations was studied in a clinical study in which 30 acne patients between 18 and 30 years of age (12 female and 18 male) were enrolled

All of them were rated as moderate or severe acne based on the number of acne lesions present when enrolling them.

Lesions on the face and neck were counted by classifying them in open and closed comedones (non-inflammatory lesions) and in Papules and Pustules (inflammatory lesions).

Participants with at least 30 non-inflammatory lesions and 30 inflammatory lesions were selected. Sebaceous secretion was determined in all participants in each observation using the method described by Harris, Downing, Stewart and Strauss¹

The participants were divided into five groups of 6 individuals each and assigned Formulas 1, 2, 3, 4 and 5 (5-A+5-B). Participants were instructed to apply the assigned formula twice a day, morning and evening, after washing their face and neck with a mild assigned soap (Dove).

Sebaceous secretion and lesion count determinations were made at Time 0 (Baseline) and after 2, 4 and 8 weeks of drug use.

The data obtained are attached and tabulated as the average of each group to each observation plus or minus the standard deviation (±SD).

Sebaceous Secretion Determination¹

| Weeks | Formula 1 | Formula 2 | Formula 3 | Formula 4 | Formula 5 |
|---|---|---|---|---|---|
| 0 (Baseline) | 3.57 ± 1.1 | 3.65 ± 1.43 | 3.26 ± 1.11 | 3.91 ± 1.52 | 3.76 ± 1.33 |
| 2 | 2.85 ± 0.70 | 3.48 ± 1.18 | 2.96 ± 0.93 | 2.74 ± 0.97 | 2.67 ± 0.91 |
| 4 | 1.14 ± 0.16 | 3.51 ± 1.35 | 1.27 ± 0.27 | 1.19 ± 0.21 | 1.10 ± 0.20 |
| 8² | 0.35 ± 0.08 | 3.23 ± 1.21 | 0.42 ± 0.07 | 0.38 ± 0.05 | 0.43 ± 0.09 |

Notes
¹Data are expressed as mg/10 cm²/3 hours
²Formulas 1, 3, 4 and 5 vs Formula 2: p < 0.01 (paired T test)

Lesion Count
COMEDONES (Closed + Open)

| | | | | | |
|---|---|---|---|---|---|
| (Base) | 47 ± 11 | 39 ± 8 | 51 ± 13 | 43 ± 7 | 53 ± 14 |
| 2 | 30 ± 7 | 34 ± 9 | 45 ± 12 | 32 ± 8 | 43 ± 10 |
| 4 | 16 ± 5 | 23 ± 9 | 40 ± 11 | 23 ± 6 | 19 ± 7 |
| 8 | 7 ± 4 | 12 ± 6 | 35 ± 13 | 9 ± 5 | 8 ± 3 |

PAPULES + PUSTULES

| | | | | | |
|---|---|---|---|---|---|
| 0 (Base) | 32 ± 13 | 36 ± 15 | 30 ± 11 | 38 ± 15 | 37 ± 16 |
| 2 | 18 ± 9 | 28 ± 13 | 27 ± 12 | 22 ± 11 | 19 ± 10 |
| 4 | 9 ± 4 | 19 ± 7 | 19 ± 8 | 17 ± 10 | 13 ± 3 |
| 8^{1,2} | 2 ± 0.5 | 13 ± 4 | 15 ± 6 | 11 ± 5 | 8 ± 0.6 |

Note:
All data are expressed as Average ± SD
¹Formulas 1 and 5 vs 2.3 and 4: p < 0.01 (Student t test).
²Formula 1 vs. 5: p < 0.05 (Student t test)

1-13 (canceled)

14. A formulation for the topical treatment of acne containing 13-cis-retinoic acid in concentrations from 0.001% to 0.25% and another retinoid with keratolytic therapeutic action in acne in a cosmetically acceptable aqueous vehicle and containing antioxidants and preservatives suitable to protect retinoids against oxidation.

15. The formulation according to claim 14, wherein 13-cis-retinoic acid is in concentrations preferably between 0.005% to 0.15%.

16. The formulation according to claim 14, wherein the other retinoid with keratolytic therapeutic action in acne is all-trans-retinoic acid in concentrations from 0.001% to 0.25%.

17. The formulation according to claim 16, wherein the all-trans-retinoic acid is in concentrations from 0.025% to 0.10%.

18. The formulation according to claim 14, further comprising an antibiotic capable of controlling P. acnes.

19. The formulation according to claim 18, wherein the antibiotic capable of controlling P. acnes is clindamycin in concentrations of 0.5% to 3%.

20. The formulation according to claim 19, wherein the clindamycin is in concentrations between 1% and 2%.

21. The formulation according to claim 18, wherein the antibiotic capable of controlling P. acnes is erythromycin or some other tetracycline in concentrations between 1% and 5%.

22. The formulation according to claim 14, further comprising a bactericidal agent.

23. The formulation according to claim 22, wherein the bactericidal agent is benzoyl peroxide.

24. The formulation according to claim 14, wherein one or more of the active ingredients are included in polymeric particles to obtain a gradual and extended release of the active ingredient (s) included in the polymeric system.

25. The formulation according to claim 14, wherein the acne is severe inflammatory acne.

26. The formulation according to claim 14, wherein the retinoid with keratolytic action is replaced by salicylic acid or another acid with keratolytic action.

27. A method for topical treatment of severe acne, the method comprising administering a formulation containing 13-cis-retinoic acid in concentrations from 0.001% to 0.25% and other retinoid with keratolytic therapeutic action in acne in a cosmetically acceptable aqueous vehicle and containing antioxidants and preservatives suitable to protect retinoids against oxidation.

28. The method according to claim 27, wherein the 13-cis-retinoic acid is in concentrations preferably between 0.005% to 0.15%.

29. The method according to claim 27, wherein the other retinoid with keratolytic therapeutic action in acne is all-trans-retinoic acid in concentrations from 0.001% to 0.25%.

30. The method according to claim 29, wherein the all-trans-retinoic acid is in concentrations from 0.025% to 0.10%.

31. The method according to claim 27, further comprising an antibiotic capable of controlling P. acnes.

32. The method according to claim 31, wherein the antibiotic capable of controlling P. acnes is clindamycin in concentrations from 0.5% to 3%.

33. The method according to claim 32, wherein the clindamycin is in concentrations between 1% and 2%.

34. The method according to claim 31, wherein the antibiotic capable of controlling P. acnes is erythromycin or some other tetracycline in concentrations between 1% and 5%.

35. The method according to claim 27, further comprising a bactericidal agent.

36. The method according to claim 35, wherein the bactericidal agent is benzoyl peroxide.

37. The method according to claim 27, wherein one or more of the active ingredients are included in polymeric particles to obtain a gradual and extended release of the active ingredient(s) included in the polymeric system.

38. The method according to claim 27, wherein the acne is severe inflammatory acne.

39. The method according to claim 27, wherein the retinoid with keratolytic action is replaced by salicylic acid or another acid with keratolytic action.
